(19) United States
(12) Patent Application Publication
(10) Pub. No.: US 2021/0251505 A1
(43) Pub. Date: Aug. 19, 2021

(54) METHOD AND BIOMEDIAL ELECTRONIC EQUIPMENT FOR MONITORING PATIENT'S CONDITION AFTER A STROKE

(71) Applicants: Kaunas University of Technology, Kaunas (LT); UAB Gruppo Fos Lithuania, Vilnius (LT); Lithuanian University of Health Sciences, Kaunas (LT)

(72) Inventors: Darius Jegelevicius, Kaunas (LT); Arunas Lukosevicius, Kaunas (LT); Vaidotas Marozas, Kaunas (LT); Andrius Petrenas, Kaunas (LT); Saulius Daukantas, Kaunas (LT); Monika Simaityte, Kaunas (LT); Daiva Rastenyte, Kaunas (LT); Vaidas Matijosaitis, Kaunas (LT); Kristina Lauckaite, Kaunas (LT); Rosita Makauskiene, Vilnius (LT); Mantas Mikulenas, Kaunas (LT); Federico Boero, Kaunas (LT); Andrea Sansalone, Kaunas (LT)

(21) Appl. No.: 17/265,821

(22) PCT Filed: Aug. 7, 2019
(86) PCT No.: PCT/IB2019/056718

§ 371 Date: Feb. 4, 2021

(30) Foreign Application Priority Data
Aug. 8, 2018 (LT) LT2018 537

(51) Int. Cl. A61B 5/0295; A61B 5/00; A61B 5/024; A61B 5/361; A61B 5/021; A61B 5/28

(52) U.S. Cl. CPC A61B 5/0295; A61B 5/4842; A61B 5/02416; A61B 5/7285; A61B 5/7275; A61B 5/361; A61B 5/02141; A61B 5/28; A61B 5/02405

(57) ABSTRACT

This invention discloses a novel method for monitoring and analyzing human cardiovascular functions and the equipment implementing the said method. It presents a portable device with the implemented measurement-analytical method that enables monitoring of heart function and selectively of brain tissue functions by measuring electrical bioimpedance along with electrocardiogram (ECG) and photoplethysmogram (PPG) measurements.

The presented method and equipment allow to measure two types of cardiovascular parameters, to process, synchronize, analyze them and thus to obtain information about the state of cardio and brain vascular activity changing over time. Also, spatial analysis is applied to the measured bioimpedance data which together with the temporal analysis data allows determining the patient's condition and how it changes after stroke.

### FIELD OF INVENTION

This invention relates to medical equipment in general and more particularly to the equipment and measurement-analysis method for monitoring stroke-induced brain and cardiovascular functional parameters and their dynamics after the stroke, by employing bioimpedance, electrocardiographic, photoplethysmographic and motion analysis.

### BACKGROUND ART

This disclosure provides a method and its hardware implementation for monitoring stroke-induced brain and cardiovascular functional parameters and their dynamics after the stroke. The invention is novel in that the post-stroke state is evaluated non-invasively by measuring two sets of parameters. The first set comprises multi-channel parameters derived from the measurement of electrical bio-impedance of the brain. The second set comprises multi-channel cardiovascular system parameters related to post-stroke condition, which are obtained from electrocardiography, plethysmography, and human motion sensors. All measurements are performed continuously and it is synchronised with the electrocardiogram. The time-varying bio-impedance parameters measured in many measurement channels are used to determine changes in brain tissue and their spatial distribution. Measurement data from different (multimodal) sources are combined and analyzed in a complex way, thus providing more information than analyzing each of the different measurements separately, in a non-synchronized manner.

U.S. Pat. No. 8,211,031B2 (published Jul. 3, 2012) discloses a device and method for measurement of physiological parameters by using the electrical impedance of brain tissue. The measurement of electrical impedance alone does not provide an evaluation of stroke-induced changes in parameters of cardiovascular system, as well as possibility to synchronize different types of measurement. There is also no information about spatial analysis of recorded data.

Patent application US20110190600A1 (published Aug. 4, 2011) describes a system of physiological sensors and a measurement method by using these sensors. The document lists different sensors (biopotential electrodes, optical detectors, temperature sensors, etc.) and provides a measurement method for those sensors. However, it does not mention the processing, synchronization and analysis algorithm of the recorded signals. It remains unclear how these sensors can be linked to the system to achieve a specific study result, since the principles of linking the data recorded by different sensors are not presented.

The above presented technical solutions have the following disadvantages:
electrical impedance or other parameters of brain tissue are measured separately and are not used in combination to provide new information. Parameters of brain tissue are not in combination with other parameters of cardiovascular system, i.e., there is a lack of versatile methods for data recording and holistic analysis;
electrical measurement alone does not allow to measure parameters associated with condition-induced blood pressure;
a system with several physiological sensors is presented, however, no integration and computation is included to provide quantitative parameters for evaluation patient's condition after a stroke;
there is no possibility to monitor the spatial distribution of the brain tissue changes over time

Our suggested invention provides a new technical solution without the above-listed drawbacks.

### SUMMARY OF INVENTION

The present invention provides a novel method and equipment for non-invasively monitoring and analysis of post-stroke changes in the human brain and recognizing functional pathologies of the cardiovascular system (e.g., paroxysmal atrial fibrillation, hypertension) associated with brain stroke. It presents a portable non-invasive device with the implemented measurement-analytical method that enables monitoring of heart function and selectively of brain tissue functions and their changes by measuring electrical bioimpedance along with electrocardiogram (ECG) and photoplethysmogram (PPG) and patient's motion measurements.

The presented method and equipment allow to measure two types of parameters:
electrical impedance of the brain and cardiovascular system parameters, as well as to process, synchronize, analyze them and thus to obtain information about the condition of cardio and brain vascular activity changes over time.
also, spatial analysis is applied to the measured bioimpedance data which together with the temporal analysis allows to determine the patient's condition after stroke and its changes over time.

### DESCRIPTION OF DRAWINGS

FIG. 1 is a schematic diagram of the cardiovascular monitoring (cardio-plethysmographic) equipment.

FIG. 2 is a schematic diagram of the equipment for monitoring and evaluating cerebral blood flow and cerebral conduction (by measurement of head electrical bioimpedance).

### DRAWINGS—REFERENCE NUMERALS

1—part of the cardiovascular monitoring (cardio-plethysmographic) equipment,
1.1—sensors
1.1.1—ECG electrodes
1.1.2—PPG sensors
1.1.3—inertial measurement unit (IMU)
1.1.4—altimeter
1.2—data recording
1.2.1—ECG recording
1.2.2—PPG recording
1.2.3—recording of motion data
1.2.4—array of synchronized data
1.3—data processing algorithms
1.3.1—recognition and characterization of atrial fibrillation arrhythmias
1.3.2—determination of pulse wave arrival time
1.3.3—indirect blood pressure measurements
1.4—data output, characterization
2—part of the equipment for monitoring and evaluating the cerebral blood flow and cerebral electrical conductivity (by measuring electrical bioimpedance of the brain)
2.1—sensors
2.1.1—bioimpedance sensors
2.1.3—scalp blood circulation sensor and cuff
2.2—data gathering
2.2.1—interconnection of electrodes (multiplexing)
2.2.2—data aquisition and processing
2.2.3—voltage sensing
2.2.4—creating and supplying electrical current
2.2.5—control device
2.2.6—synchronized data array
2.3—data processing algorithms
2.3.1—analysis of temporal parameters
2.3.2—analysis of spatial parameters

The presented figures are illustrative only, the scale, proportions and other aspects do not necessarily correspond to the actual technical solution.

### DETAILED DESCRIPTION OF PREFERRED EMBODIMENTS

This description provides a method and equipment for monitoring the condition of a person who has suffered a stroke and for predicting potential risks. A new technical solution can be used to detect neurophysiological changes, to detect atrial fibrillation, increased variability of blood pressure parameters, and to assess the risk of the secondary stroke. The equipment can be used both for continuous monitoring and for episodic monitoring. The equipment can be used in hospitals or other medical facilities, as well as in patient's home, or other similar environments.

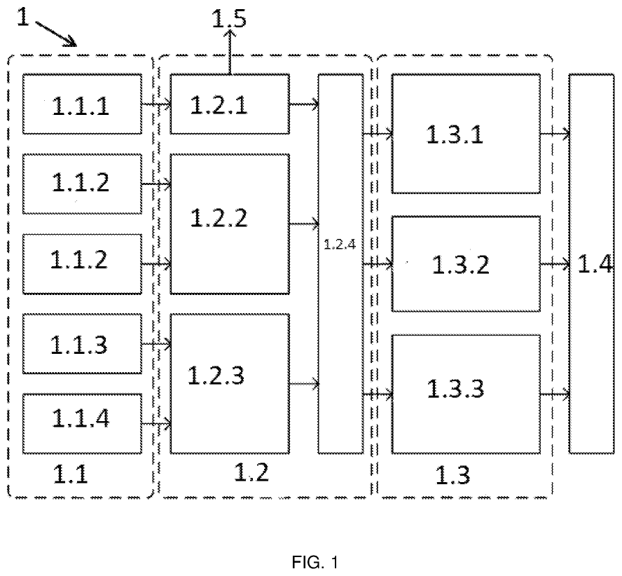

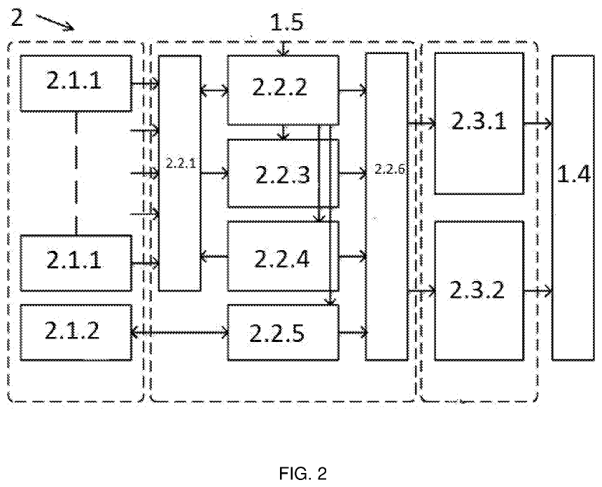

This technical solution and the equipment consists of least these two components or functional units:
part of the equipment for cardiovascular monitoring (cardio-plethysmographic) (1), (FIG. 1);
part of equipment for monitoring and evaluation of cerebral blood flow and cerebral electrical conductivity (by measuring of electrical impedance of the brain) (2) (FIG. 2)

The bioimpedance part (2) is used to measure the electrical resistance of the scalp and brain tissues. Such equipment has bioimpedance sensors (2.1.1) which are positioned closely onto the head. One pair of electrodes emits an electrical signal of a certain frequency and intensity which are generated in the current supplying device (2.2.4), and the other pair of electrodes receives the signal and transmits it to a the voltage measurement device (2.2.3) where the impedance of the scalp and brain tissues is calculated. Several pairs of such electrodes may be used. In the present invention, not only the temporal characteristics of electrical signals (2.3.1), synchronized with the cardiac cycles seen in the electorcardiogram (1.1), are measured and analyzed, but also the spatial characteristics of brain tissues (2.3.2). In this case, the spatial characteristics mean that the measurements are intended to determine the impedance distribution in the brain tissue. According to the present invention, the bioimpedance part (2) has at least the following basic functional components: sensors (2.1), data reception (2.2.2), data processing (2.2.2) and data output, visualization (1.4). One pair of electrodes delivers a predefined frequency alternating current signal that penetrates through the head tissue and creates a potential difference recorded by another pair of electrodes. The recorded signal is transmitted to the functional part of the data gathering (2.2), where the electrical signals are processed, synchronized with (1.1.1) the ECG sensor signal, thus forming a data array (1.2.4) which is transmitted to the functional part of data analysis (2.3). In the data processing functional part (2.3), array of the recorded data (1.2.4) are analyzed to determine the temporal and spatial distribution characteristics of the bioimpedance. The result of the array (1.2.4) processing is represented by the functional part of the result visualization (1.4).

To collect signals, the device employs several methodologies and combines adjacent and opposite methodologies, by using their opposite properties to estimate potential difference at the surface and depths of the brain tissue. The device also can capture signals using techniques of intersecting and all possible positioning, avoiding repetitive or reversed polarity measurements.

The device reduces the effects of scalp blood flow noise by using a cuff to block the blood flow to the scalp before measurement, and a photoplethysmographic sensor to monitor the success of the blood flow blocking, which gives a signal that the cuff is already tightened.

The device for tomographic measurements of brain impedance can use a different frequency of the supplied current to control the current path at the cellular level of head tissues.

The device can perform reoencephalography measurements of temporal changes in cerebral blood flow, along with impedance, ECG and PPG measurements.

Signal strength is controlled by passive and active methods. The signal is amplified in the analog-to-digital converter (ADC), thus increasing the power of the useful signal. The signal is also amplified by increasing the current amplitude to a maximum permissible level depending on the frequency (10 mA at 100 kHz).

For impedance scanning, the device uses 24-bit ADC increasing the dynamic range of measurements.

The device has two configurations of 16 and 32 channels providing different measurement accuracy.

As mentioned above, the bioimpedance system (2) measures the impedance of head tissues, therefore, the implementation of this signal measurement system part has to be adapted to fit it onto the head. The shape may resemble a hat, helmet or similar.

The cardiovascular monitoring part (1) is designated to assess the patient's condition after a stroke because this part (1) is related to cerebral blood circulation. Cardiovascular monitoring (1) allows to measure parameters such as atrial fibrillation, pulse rate, and blood pressure, which are important for assessing the patient's condition after a stroke. The recorded electrocardiogram is also used to synchronize data between the two parts of the system (1.1). The cardiovascular monitoring portion (1) can be implemented as a wearable wristband.

The cardiovascular monitoring part (1) consists of at least the following basic functional components: sensors (1.1), data acquisition components (1.2), data processing (1.2), and result visualization tools (1.4). The functional part of the sensors consists of several types of sensors: electrocardiogram (ECG) sensors (1.1.1), at least two photoplethysmography (PPG) sensors (1.1.2), inertial motion sensors (1.1.3) (accelerometers, gyroscopes), altimeter (height sensor) (1.1.4). The recorded signals by all sensors are transmitted to the functional part of data receiving (1.2.1), where the signals are filtered, amplified, digitized, synchronized to the electrocardiogram signal (1.5) and further transmitted to the functional part of data processing (1.3). In the functional part of data processing (1.3), data is processed to detect atrial fibrillation (1.3.1), measure pulse wave arrival (1.3.2) and indirectly evaluate changes in blood pressure (1.3.3). The result of data processing is represented by the functional part of the result representation (1.4), which can also be used to represent bioimpedance data.

The method implemented in the equipment comprises at least the following operating steps:
measuring the electric bioimpedance of the head comprising data set Z(x,y,t,f), in the measurement part (2);
measuring the data for cardio-plethysmographic dataset ECG-PPG(t), in cardio-plethysmographic part (1);
synchronization of impedance data cardio-plethysmographic data set with an electrocardiogram (1.5) to obtain an array of synchronized data (1.2.4);
applying adaptive data processing and analysis algorithms for the synchronized data array (1.2.4), such as:
   truncated Landweber iteration scheme is applied for the evaluation of spatial distribution of the bioimpedance;
   the variability of blood pressure parameters is evaluated by the variability of pulse wave arrival time measured by using the wristband;
   detection of atrial fibrillation episodes is based on the evaluation of pulse irregularity;
   the concentration of arrhythmia episodes is evaluated by the episode aggregation or density parameter.
present the processed and analyzed data by the data display means (1.4) and, if necessary, provide for further processing or presentation.

The above computational steps are implemented by electronic computing means connected to the equipment parts that measure the above listed physical parameters, as well as connected by communication means to remote electronic computing devices. The aforementioned electronic computing means are:
electronic devices with a processor (processors) for processing data,
cache and/or permanent storage devices for storing data and information,
internal communication means for communication between components,
communication devices with external devices.

Aforementioned means may be realized as a computer or a microcontroller with a special software implementing the above-listed computation steps. The electronic equipment implementing analysis and computation may be located at a distance from the measuring equipment. The aforementioned equipment may be portable.

To illustrate and describe the present invention, the following description of the preferred embodiments is provided above. It is not a detailed or restrictive description to determine the exact form or embodiment. The description above should be viewed as an illustration rather than a limitation. Many modifications and variations may be apparent to those skilled in the art. For those skilled in the art, an embodiment is selected and described in order to allow to understand the principles of the invention and its best practical application for different embodiments with different modifications suitable for a particular application or implementation application. The scope of the invention is defined by claims and its equivalents, in which all the foregoing terms have the widest possible meaning unless otherwise stated.

Embodiments described by those skilled in the art can be made with amendments and modifications, without departing from the scope of the present invention as defined by Claims of the invention.

1. A method of monitoring and predicting a patient's condition after a stroke, comprising steps of:
recording head electrical bioimpedance data to form an impedance data set Z(x, y, t, f);
recording cardio plethysmographic data comprising ECG(t) and FPG(t) cardio-plethysmographic data sets;
further comprising:
synchronizing aforementioned impedance and cardio-plethysmographic data sets to an electrocardiogram, resulting in an array of synchronized data;
applying data processing and analysis algorithms to said array of synchronized data;
presenting said processed and analyzed data by data display means; and
transmitting said processed and analyzed data for further processing or presentation.

2. The method of monitoring the patient's condition after a stroke according to claim 1, characterized in that the measured data of the head electrical bioimpedance is processed to locate stroke-induced changes in brain tissue and function and also including:
recording changes in the patient's cardiovascular system represented by ECG(t), PPG(t), and derivated parameters including Heart Rate (HR), Heart Rate Variability (HRV), Pulse Arival Time (PAT), PAT variability, and Atrial Fibrillation (AF); and
relating the state of the patient's cardiovascular system with the state of the cerebral state.

3. The method of monitoring the patient's condition after a stroke according to claim 1, characterized in that at least one algorithm of estimating the blood pressure variability associated with stroke is based on the simultaneous use of electrocardiographic and photoplethysmographic signals, recorded by a device worn on the patient's wrist.

4. The method of monitoring the patient's condition after a stroke according to claim 1, characterized in that the impedance signals are collected by using a combination of opposite (with current electrodes facing each other) and adjacent (with current electrodes being adjacent) current electrode measurements.

5. The method of monitoring the patient's condition after a stroke according to claim 1, characterized in that the head electrical bioimpedance data is enhanced (increased signal to noise ratio) by using an inflatable cuff positioned around the patient's head and below impedance electrodes with a built-in photoplethysmographic sensor tracking the blood flow and giving a signal that the cuff was tightened enough.

6. The method of monitoring the patient's condition after a stroke according to claim 1, characterized in that the head electrical bioimpedance data is used to calculate spatial impedance distribution, related to brain tissue and cerebral blood circulation, by using electric currents of different frequencies.

7. The method of monitoring the patient's condition after a stroke according to claim 1, characterized in that the head electrical bioimpedance data is processed to form a rheoencephalographic signal of the cerebral blood flow, together with spatial impedance and temporal signals of ECG and PPG.

8. The method of monitoring the patient's condition after a stroke according to claim 1, characterized in that the strength of the impedance measurement signal is controlled by a supply current depending on the frequency of the supplied current and amplification of the signal.

9. Equipment for monitoring and predicting the patient's condition after a stroke, configured to collect data required for the method according to claim 1, further comprising:
two time-synchronized subsystems:
1) a head bioimpedance subsystem with surface electrodes and electronics acquiring signals of the impedance temporally and spatially together with equipment for reduction of scalp blood flow noise on impedance signals by reducing the scalp blood flow; and
2) a cardiovascular monitoring subsystem with electrocardiographic (ECG) and photoplethysmographic (PPG) sensors.
